(19) United States
(12) Patent Application Publication
(10) Pub. No.: US 2005/0202562 A1
(43) Pub. Date: Sep. 15, 2005

(54) Volumetric Karl-Fischer titration with stabilized solvent

(76) Inventors: Larry M. Girdler, Cincinnati, OH (US); Gene Desotelle, Mason, OH (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/069,055

(22) Filed: Mar. 2, 2005

Related U.S. Application Data
(60) Provisional application No. 60/548,875, filed on Mar. 2, 2004.

(51) Int. Cl. G01N 33/18

(52) U.S. Cl. 436/42

(57) ABSTRACT

Described are methods for avoiding false low water determinations in a volumetric Karl-Fischer water determination by including an amine group-containing compound as a stabilizer in the solvent charged to the reaction vessel separate from the titration. Also, described are solvent/stabilizer compositions for such methods.

This application claims the benefit of the filing date of U.S. Provisional Application Ser. No. 60/548,875 filed Mar. 2, 2004, which is incorporated by reference herein.

The invention described herein includes an improved method for performing volumetric Karl-Fischer titration. The method involves use of a solvent added separately from the titrant and stabilization of this solvent with a stabilizing agent. The stabilizing agent is preferably an amine group-containing compound. The inventions described further include, for example, compositions which are a stabilized solvent for use in volumetric Karl-Fischer titrations which compositions contain a particular solvent and particular type and amount of amine group-containing compound as stabilizer.

Karl-Fischer titration methods, apparatus and reagents are known in the art. Volumetric Karl-Fischer titration is used to assess the water content of samples by accurate titrimetric methods. Assessing water content of samples is important to many industries, for example, the food, pharmaceutical, polymer, biotechnology and cosmetic industries, and for a variety of types of samples, for example, organic and inorganic chemicals, fats and oils.

The methods are based on the oxidation of sulfur dioxide by iodine in the presence of water according to the following chemical equations:
2H₂O+SO₂+I₂⇄H₂SO₄+2HI
ROH+SO₂+RN→(RNH).SO₃R
(RNH).SO₃R+I₂+H₂O→(RNH).SO₄R+2(RNH)I

Several types of methods are known which include several types of volumetric Karl-Fischer titration and several types of coulometric Karl-Fischer titration. In volumetric Karl-Fischer titrations, typically after steps for standardizing the system, a solution containing sulfur dioxide and a known concentration of iodine is titrated into a the sample to be determined. The iodine oxidizes the sulfur dioxide using stoichiometric amounts of water in the sample. The point where all the water is consumed can be determined by consequent excess of iodine by visual, photometric or electromagnetic means. At this point, the volume of titrant with known iodine concentration used is determined which, by the stoichiometric equation, indicates the amount of water in the sample. Potentiometric recognition or dead stop indication provides a very precise calculation of the endpoint. Potentiometric recognition is particularly preferred. Systems for making such Karl-Fischer volumetric titrations are commercially available, for example, one commercially known system is a Mettler Toledo DL38 Titrator. Methods for volumetric Karl-Fischer titration are described, for example, in U.S. Pat. Nos. 4,378,972 and 4,429,048 to Scholz, U.S. Patent Application Publication No. 2002/0127726 to Hoffman and EP 127,740. There are a number of different arrangements possible for volumetric Karl-Fischer titration, as exemplified in these documents. The disclosures in these documents regarding background of the Karl-Fischer methods, nature of the titrant, manner of titrating and determining water content thereby are incorporated herein by reference.

The above-mentioned Scholz patents disclose that it was known to use pyridine as an amine in the titration agent and that their invention relates to using an aliphatic amine containing oxygen atoms (such as ethanolamines) or a nitrogen-containing heterocyclic compound (such as imidazoles) in the titration agent. The Hoffman published application discloses that the amine-containing base component in the titration agent takes part in the reaction of the iodine, sulfur dioxide and water by neutralizing the acid formed to facilitate a quantitative reaction. Hoffman's purported invention was to use a combination imidazole and substituted imidazole in the titrant as such base to avoid the formation of imidazolinium salts which occurred, particularly in high temperature/humidity environments, when using imidazole alone as the base.

In one arrangement for volumetric Karl-Fischer titration, the system (see FIG. 1) provides a reaction vessel which is equipped with a means for determining the endpoint of the above-described titration of iodine-sulfur dioxide into a water-containing sample and a means for titrating a titrant into the reaction vessel. In this arrangement, the titrant contains the iodine and sulfur dioxide, an anhydrous solvent (preferably alcohol-containing) and, optionally, an amine compound. Further, the reaction vessel is provided with a solvent before the titration occurs. This solvent is preferably an alcohol-containing solvent. In operation of such arrangement, the solvent is charged into the reaction vessel but the solvent typically contains some amount of water and this must be zeroed-out so as not to effect the sample calculation. This is done by titrating titrant into the solvent until the endpoint is determined, indicating that all the water has been reacted. The instrument is then ready for sample analysis. But, typically, the instrument is first standardized using solutions of known water content, such as pure water and/or solutions of solvent with a known water content. By testing of such standard samples, the instrument can be calibrated for accurate reading. Once the standard is titrated to the endpoint, the instrument is to the zeroed-out point again and ready for another standard or sample. Similarly, after a sample is titrated to the endpoint to determine its water content, the instrument is zeroed-out. Thus, a number of subsequent samples can be subject to water determination by titration without re-setting of the instrument. Additional titrations can be conducted until:
the reaction vessel becomes too full of solution,
the solution in the reaction vessel turns too dark from excess iodine, for example, making determination of the endpoint difficult,
the nature of the samples to be tested is such that undesired side reactions would be expected (e.g., aldehyde or ketone-containing samples are desired to be tested after hydrocarbon-containing samples, in which case a methanol-free solvent is needed),
the results are of an unexpected nature indicating, for example, that some fouling of the system has occurred.

As an aspect of the invention herein, it has been discovered that, when using the above-described arrangement with: (1) a titrant containing iodine, sulfur dioxide, an amine base and an alcohol solvent and (2) an alcohol solvent charged to the reaction vessel, a problem effecting accuracy of water content readings occurs under certain circumstances. Particularly, after an instrument has been charged with the solvent and zeroed-out or zeroed-out as a result of titration of a standard or sample, and then the instrument is left to sit for a period of time, e.g., several hours or overnight, the subsequent titration conducted often results in a false low water content reading. This is obviously highly undesirable as it may lead to false water content readings of samples which could have serious consequences or requires a time-consuming re-standardizing of the instrument after prolonged periods of non-use. The invention here was made as a result of investigation into a way for avoiding this undesired result.

It was discovered that addition of a stabilizer to the solvent charged into the reaction vessel will eliminate the problem of false low water readings after a period of non-use of a Karl-Fischer instrument which has already been used for some titration, i.e., either for zeroing-out water, standardizing and/or determining samples. Thus, the invention includes methods for avoiding false low water determinations in a volumetric Karl-Fischer water determination, which comprises: in a method wherein an alcohol-containing solvent is charged to a reaction vessel and a titrant comprising iodine, sulfur dioxide, a titrant solvent and, optionally, a titrant base compound, is subsequently titrated into the reaction vessel to:
zero-out the water contained in the alcohol-containing solvent charged to the reaction vessel;
determine the water content of a standard of known water concentration charged into the reaction vessel containing alcohol-containing solvent; and/or
determine the water content of a sample charged into the reaction vessel containing alcohol-containing solvent;

including an amine group-containing compound, separate from the titration, as a stabilizer in the solvent charged to the reaction vessel. The invention also includes compositions of solvent/stabilizer for carrying out these methods.

The stabilizer is preferably provided in the solvent charged to the reaction vessel in an amount of 2 to 15%, more preferably 3 to 7%, by weight based on the total resulting composition of solvent and stabilizer. The stabilizer is preferably provided in the solvent before the solvent is charged into the reaction vessel. But it may be added to the reaction vessel after the solvent. The stabilizer can even be added after any of the steps of titrating to:
zeroing-out the water contained in the alcohol-containing solvent charged to the reaction vessel;
determining the water content of a standard of known water concentration charged into the reaction vessel containing alcohol-containing solvent; and/or
determining the water content of a sample charged into the reaction vessel containing alcohol-containing solvent;
   as long as the stabilizer is added before a prolonged period, e.g., an hour or more, of inactivity of the system. Inactivity meaning periods where no titration is conducted. Thus, the invention is particularly suited to methods, wherein, after the stabilizer is provided in the solvent, and, after at least one titration step to:
zero-out the water contained in the alcohol-containing solvent charged to the reaction vessel;
determine the water content of a standard of known water concentration charged into the reaction vessel containing alcohol-containing solvent; and/or
determine the water content of a sample charged into the reaction vessel containing alcohol-containing solvent;
   the reaction vessel is maintained inactive for at least one hour, e.g., several hours or overnight, and then a further titration step to:
zero-out the water contained in the alcohol-containing solvent charged to the reaction vessel;
determine the water content of a standard of known water concentration charged into the reaction vessel containing alcohol-containing solvent; and/or
determine the water content of a sample charged into the reaction vessel containing alcohol-containing solvent;
   is conducted.

The stabilizer is an amine-containing compound. This encompasses acyclic and cyclic amines, which may have 1, 2 or 3 oxygen atoms, preferably in the form of hydroxy groups. Included are acyclic primary amines having from 2 to 6 carbon atoms and optionally 1, 2 or 3 hydroxy groups. Examples include: morpholine, piperidine, piperazine, n-propylamine, isopropylamine, diethylamine, triethylamine, dimethylamino-propylamine, ethanolamine, diethanolamine, triethanolamine, mono-, di- or tri-isopropanolamine, tris(hydroxymethyl)-aminomethane or guanidine. Included as cyclic amines are heterocyclic compounds having five or six ring members, including at least one nitrogen, which are optionally substituted, for example, by 1, 2 or 3 alkyl radicals having from 1 to 4 carbon atoms, or by 1, 2 or 3 phenyl radicals or a benzo group. The heterocyclic amines preferably contain at least 2, preferably 2 or 3, hetero-atoms, one of which at least is a nitrogen atom. Particularly preferred are five-membered, optionally substituted, heterocyclic compounds having 2 nitrogen hetero-atoms in the ring, especially imidazoles or derivatives thereof. Other examples of heterocyclic amines for the stabilizers include: imidazole, 1-methylimidazole, 1-ethylimidazole, 1-propylimidazole, 1-butylimidazole, 2-methylimidazole, 2-ethylimidazole, 2-propylimidazole, 2-butylimidazole, 4-methylimidazole, 4-butylimidazole, 1,2-dimethylimidazole, 1,2,4-trimethylimidazole, 1-phenylimidazole, 2-phenylimidazole and benzimidazole, furthermore imidazoline, 2-methylimidazoline (lysidine), 2-phenylimidazoline, and thiazole, 2-methylthiazole, 2-ethylthiazole, 4-methylthiazole, 4-ethylthiazole, 2-phenylthiazole, 4-phenylthiazole, benzothiazole, pyrimidine, 4-methylpyrimidine, 4-ethylpyrimidine, 1,3,5-triazine and 1,2,4-triazine.

Most preferably, the stabilizer is an alkanolamine, a substituted or unsubstituted pyridine or derivative thereof, a substituted or unsubstituted imidazole or derivative thereof or a mixture of any of the above. By substituted is intended one or more alkyl (e.g., methyl, ethyl, propyl or butyl), aryl (e.g., phenyl) or aralkyl (e.g., benzyl) substituents or combinations of such substituents on the pyridine or imidazole ring. By derivative thereof is meant any of the above substituted or unsubstituted pyridine or imidazole compounds which is derivatized to make the amine a quaternary amine salt, some other type salt of the amine, an acid of the amine (e.g., with Cl, I, or Br halogens), an ester (e.g., benzoate of imidazole). Particularly preferred as the stabilizer is: mono-, di- or tri-ethanolamine; mono-, di- or tri-isopropanolamine; imidazole; 2-methylimidazole; 2-ethyl-4-methylimidazole; 2-ethylimidazole; phenylimidazole; benzimidazole; pyridine; or any mixture thereof.

The alcohol-containing solvent charged to the reaction vessel is preferably methanol, ethanol, a glycol ether (such as diethylene glycol monomethyl or monoethyl ether), 2-methoxyethanol, a mixture of a primary amount of one of these with another solvent (particularly chlorinated solvents) or a mixture of any of the above. Most preferably, the solvent is methanol or primarily methanol with an additive. The selection of solvent will depend on its ability to dissolve and compatibility with the stabilizer, the titrant and any samples tested. For example, a solely methanol solvent cannot be used when determining samples containing aldehydes or ketones due to side reactions. Additional solvents used with the alcohol-containing solvent are added to improve its dissolution or compatibility properties. For example, it is useful to add chloroform to a methanol solvent to aid in dissolving fats, oils and long chain hydrocarbons. Dimethylformamide (DMF) is also a useful additive in the solvent. A mixture of methanol with chloroform and DMF can also be useful.

The invention can be practiced in conjunction with any type of titrant known to be used in such volumetric Karl-Fischer titrations. Examples are provided in the above-referenced patents/applications. Preferably, the invention is conducted in conjunction with a titrant that comprises iodine, sulfur dioxide, an alcohol solvent and, an amine-containing base compound. Typically, the alcohol solvent in the titrant is a glycol ether solvent and the base compound is an amine such as defined in U.S. Pat. No. 4,429,048 (incorporated herein by reference). The concentration of the iodine in the titrant and the amount titrated is used to determine the water content in the sample being titrated. The iodine content in the titrant is preferably from 1 to 5 mg/ml of titrant.

The method of titration is carried out according to known methods, for example, as described above. It is preferred if the endpoint of titration is determined potentiometrically. Using the known instruments, the titration and water content determination is preferably automated.

The method according to the invention is advantageous, for example, because zeroing-out steps and/or titrations can be conducted and then the system can be left inactive for several hours, overnight, or even longer, and then further titrations resumed without occurrence of false low water readings. This avoids the occurrence of an erroneous reading or the need to re-set the system after inactivity. Demonstration of this advantage is provided in the following examples.

The entire disclosure of all applications, patents and publications, cited herein is incorporated by reference herein.

### BRIEF DESCRIPTION OF THE DRAWINGS

FIG. 1 is schematic representation of an instrument for conducting methods according to the invention.

FIGS. 2-12 are graphs of the data of water recovery % values from the tests described in Examples 1-15 below.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### Example 1

### (Not According to Invention)

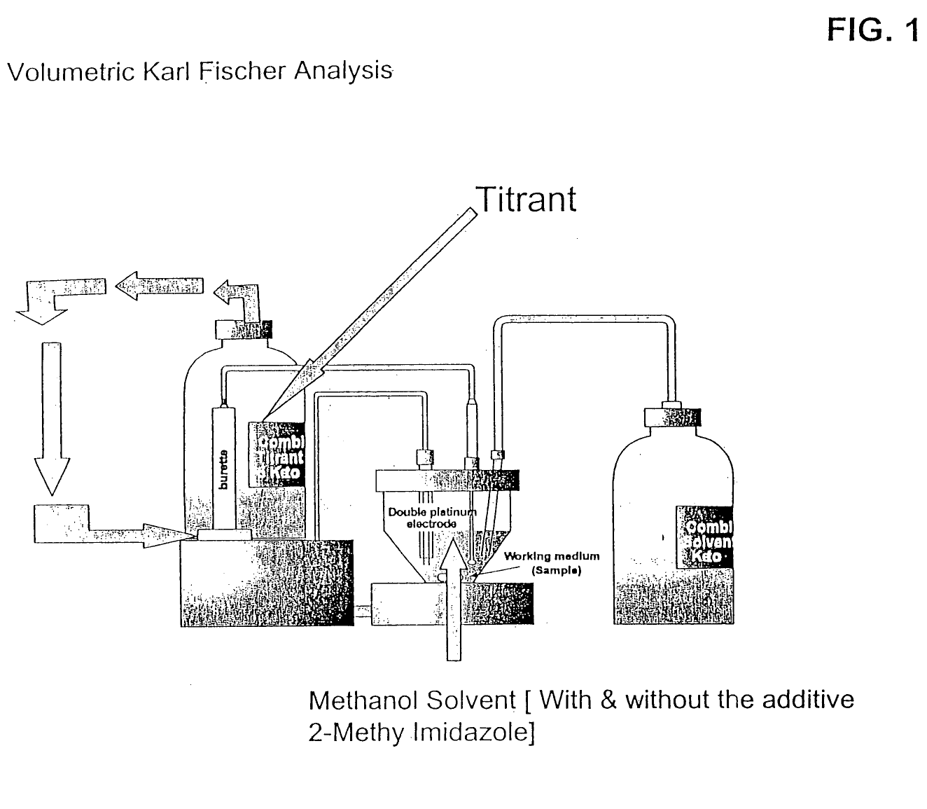

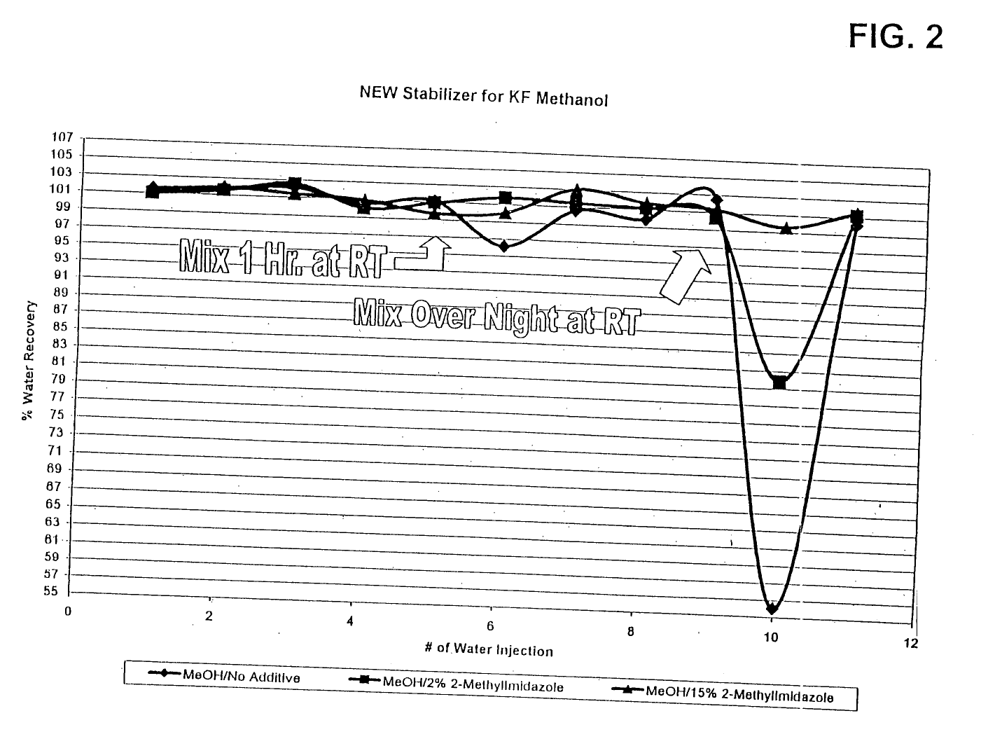

Using a known volumetric Karl-Fischer titration system (Mettler Toledo DL38 Titrator) schematically shown in FIG. 1, Karl-Fischer titrations are performed. A methanol solvent, for example of 50 to 100 ml, is charged to the reaction vessel. The solvent is zeroed-out, i.e., blanked, by titration so that it has a zero water value. Five pure water samples, 0.0200 grams each, are successively injected into the reaction vessel and titrated with a known titrant (i.e., Diethylene Glycol Monoethyl Ether (CAS 111-90-0) 50-75%, Sulfur Dioxide (CAS 7446-09-5) 0-10%, Iodine (CAS 7553-56-2) 10-20%, Imidazole (CAS 288-32-4) 10-20%) using the automated instrument. The instrument is then left inactive for one hour. A further pure water sample of 0.0200 grams is injected and titrated in the same manner and then three further identical samples are injected and titrated. The instrument is left inactive overnight (16 hours) and the next morning another identical water sample is titrated in the same manner and then a further sample immediately following. Thus, 11 samples total are injected and titrated. The water recovery % by titration for each sample is determined and recorded in FIG. 2 (shown by the line with diamond-shaped points). The water recovery values determined are at or near the expected 100% water recovery value except the 6^{th} sample injected immediately after the 1 hour inactivity has a false low value about 96% and the 10^{th} sample injected immediately after the overnight activity has a false very low value about 56%.

### Example 2

The same procedure of Example 1 is repeated except that the methanol solvent charged to the reaction vessel additionally contains 2% by weight of a 2-methylimidazole stabilizer. The water recovery values are shown also in FIG. 2 by the line with the square-shaped points. It is seen that the stabilizer eliminates the false low water recovery value of the 6^{th} injection, after one hour inactivity, and lessens the extent of the false low water recovery value of the 10^{th} injection, after overnight (about 16 hours) inactivity.

### Example 3

The same procedure of Examples 1 and 2 is repeated except that the methanol solvent charged to the reaction vessel contains 15% by weight of a 2-methylimidazole stabilizer. The water recovery values are shown also in FIG. 2 by the line with the triangle-shaped points. It is seen that the stabilizer eliminates the false low water recovery value of the 6^{th} injection, after one hour inactivity, and the false low water recovery value of the 10^{th} injection, after overnight inactivity.

### Example 4

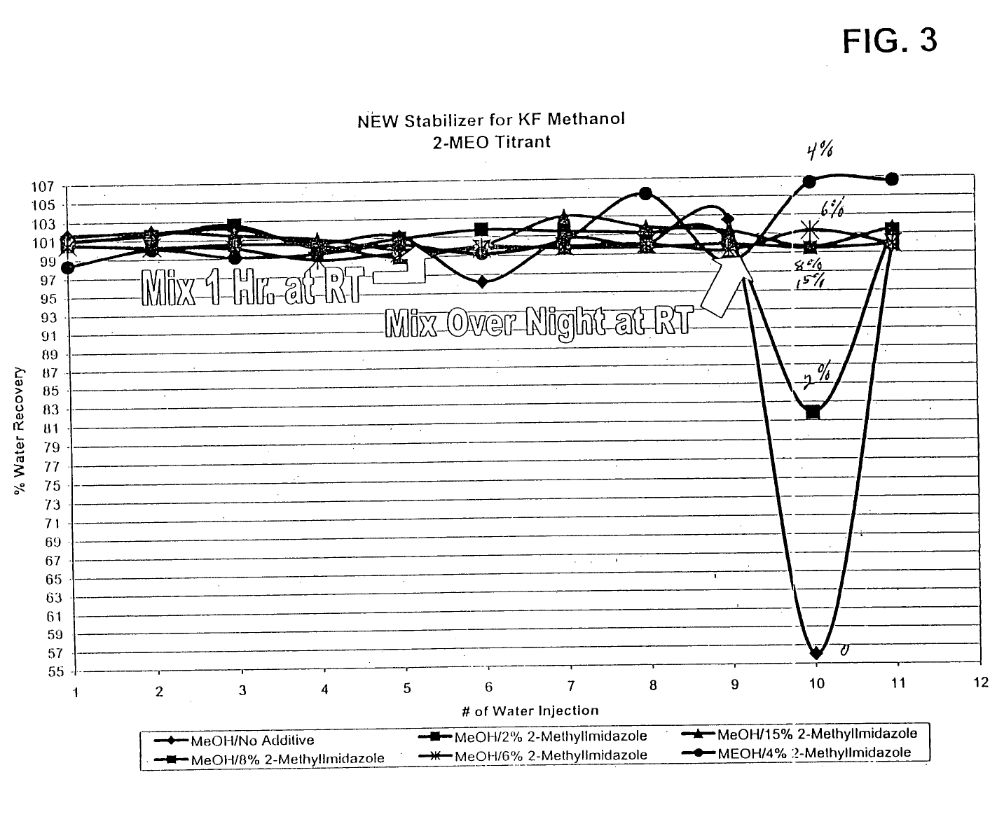

The same procedure of Examples 1-3 is repeated three more times except that the methanol solvent charged to the reaction vessel contains 4, 6 and 8%, respectively, by weight of the 2-methylimidazole stabilizer. The water recovery values are shown in FIG. 3 by the lines with the circle-, star- and square-shaped points. It is seen that, in each case, the stabilizer eliminates the false low water recovery value of the 6^{th} injection, after one hour inactivity, and the false low water recovery value of the 10^{th} injection, after overnight inactivity.

### Example 5

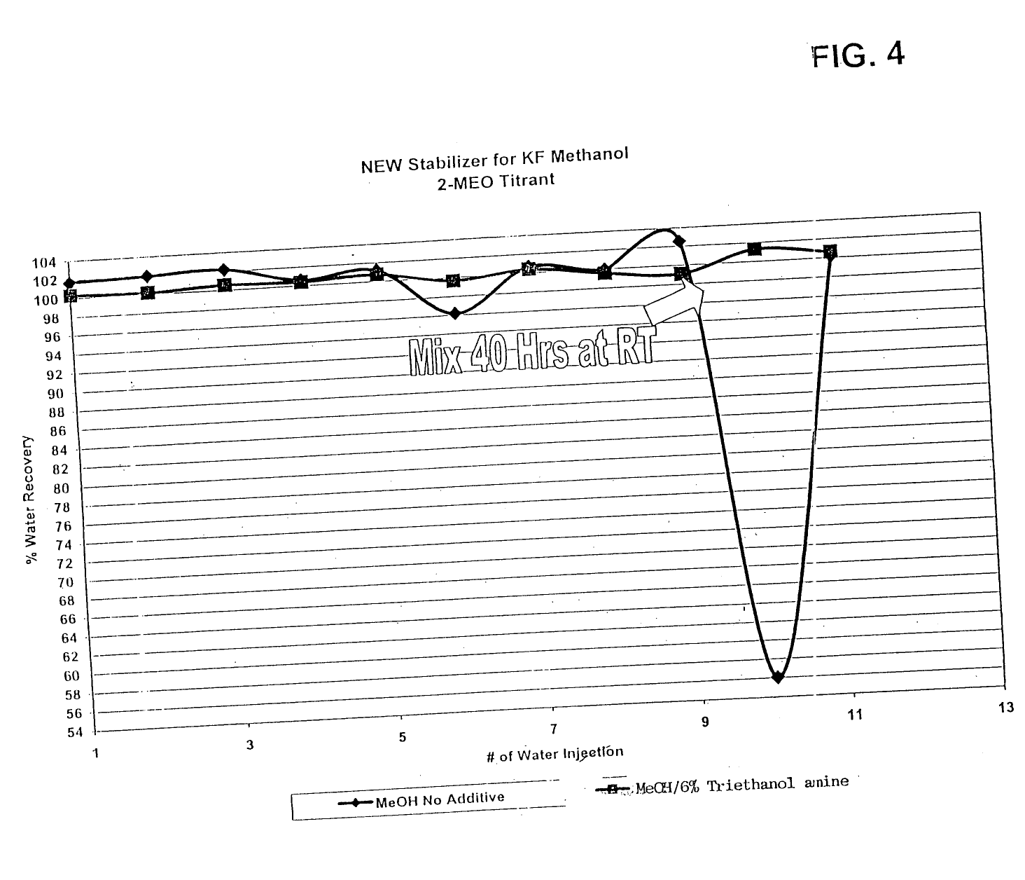

The same procedure of Examples 1-3 is repeated except that the methanol solvent charged to the reaction vessel contains 6% by weight of triethanolamine as the stabilizer. Also, the time period between the 9^{th} and 10^{th} injections is extended to 40 hours. The water recovery values are shown in FIG. 4 by the lines with the square-shaped points and the data from the previous run using no stabilizer in the methanol solvent is included for comparison purposes. It is seen that the stabilizer eliminates the false low water recovery values for titrations conducted after both a 1 hour and 40 hour period of inactivity.

### Example 6

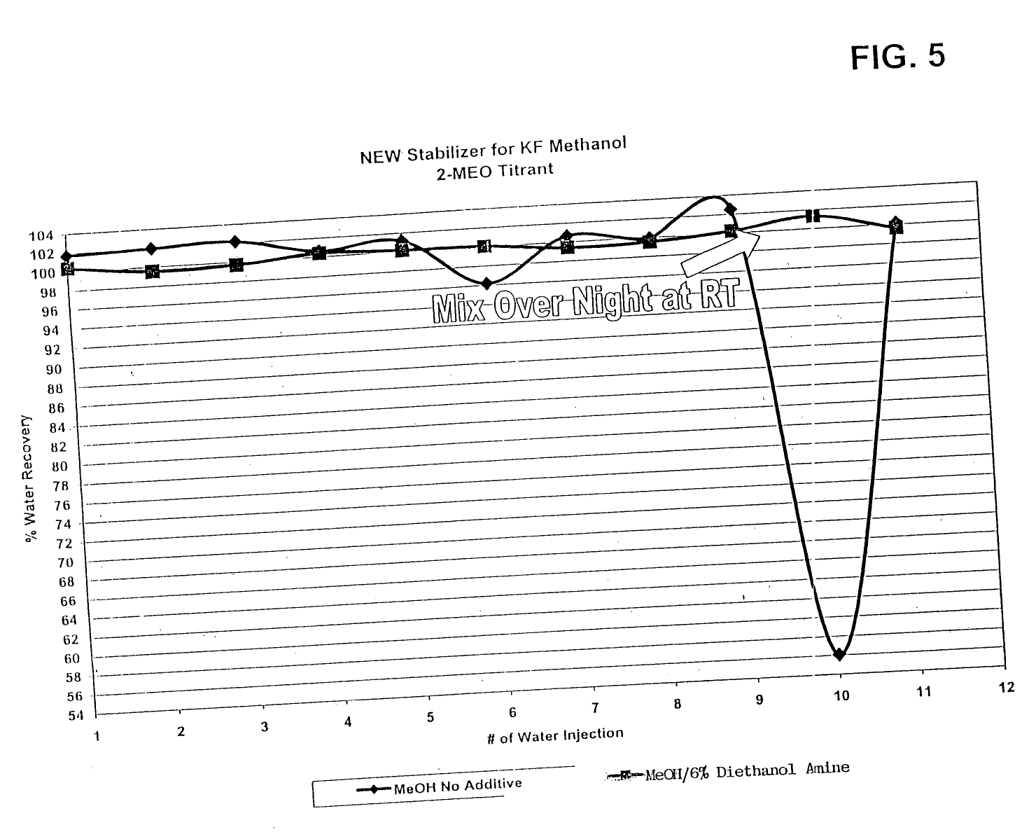

The same procedure of Examples 1-3 is repeated except that the methanol solvent charged to the reaction vessel contains 6% by weight of diethanolamine as the stabilizer. The water recovery values are shown in FIG. 5 by the lines with the square-shaped points and the data from the previous run using no stabilizer in the methanol solvent is included for comparison purposes. It is seen that the stabilizer eliminates the false low water recovery values for titrations conducted after both a 1 hour and 16 hour period of inactivity.

### Example 7

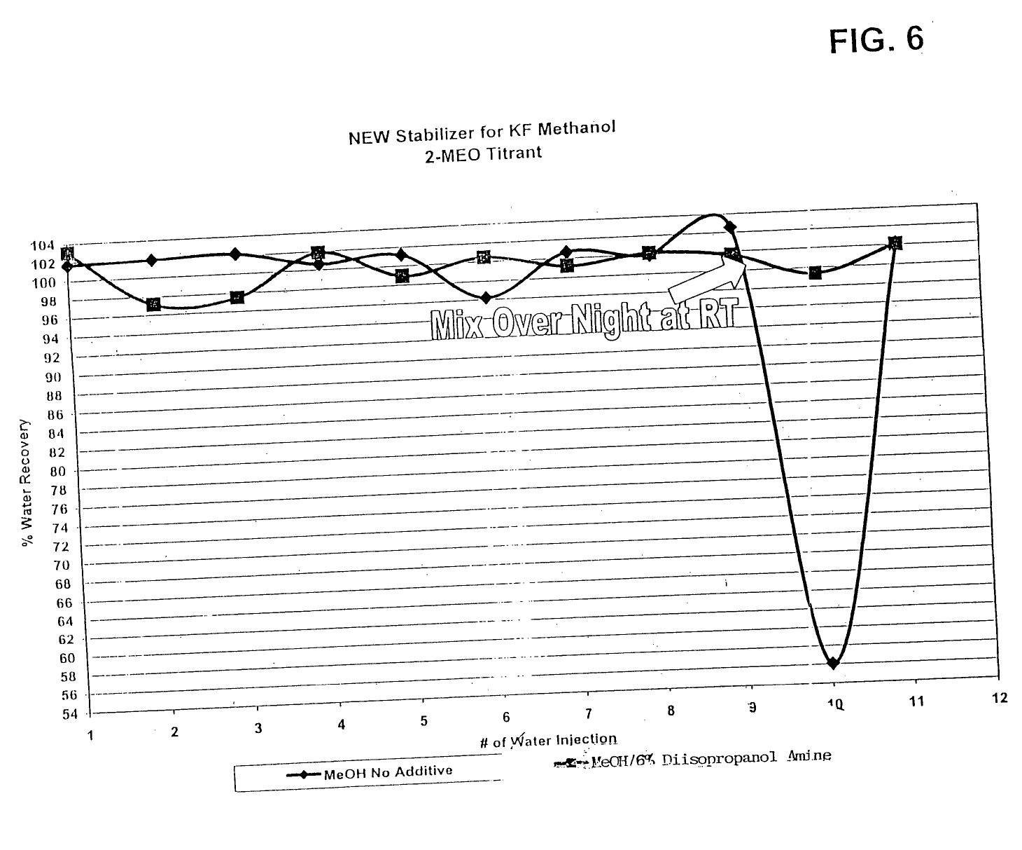

The same procedure of Examples 1-3 is repeated except that the methanol solvent charged to the reaction vessel contains 6% by weight of diisopropanolamine as the stabilizer. The water recovery values are shown in FIG. 6 by the lines with the square-shaped points and the data from the previous run using no stabilizer in the methanol solvent is included for comparison purposes. It is seen that the stabilizer eliminates the false low water recovery values for titrations conducted after both a 1 hour and 16 hour period of inactivity.

### Example 8

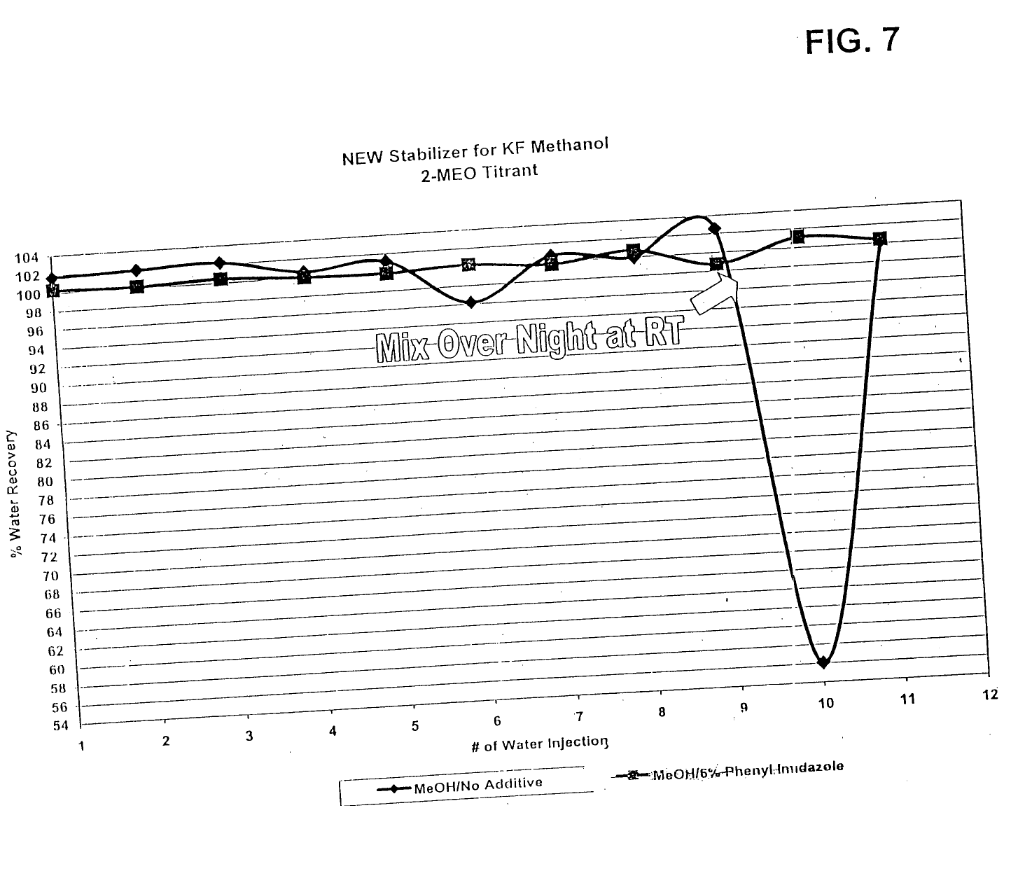

The same procedure of Examples 1-3 is repeated except that the methanol solvent charged to the reaction vessel contains 6% by weight of phenylimidazole as the stabilizer. The water recovery values are shown in FIG. 7 by the lines with the square-shaped points and the data from the previous run using no stabilizer in the methanol solvent is included for comparison purposes. It is seen that the stabilizer eliminates the false low water recovery values for titrations conducted after both a 1 hour and 16 hour period of inactivity.

### Example 9

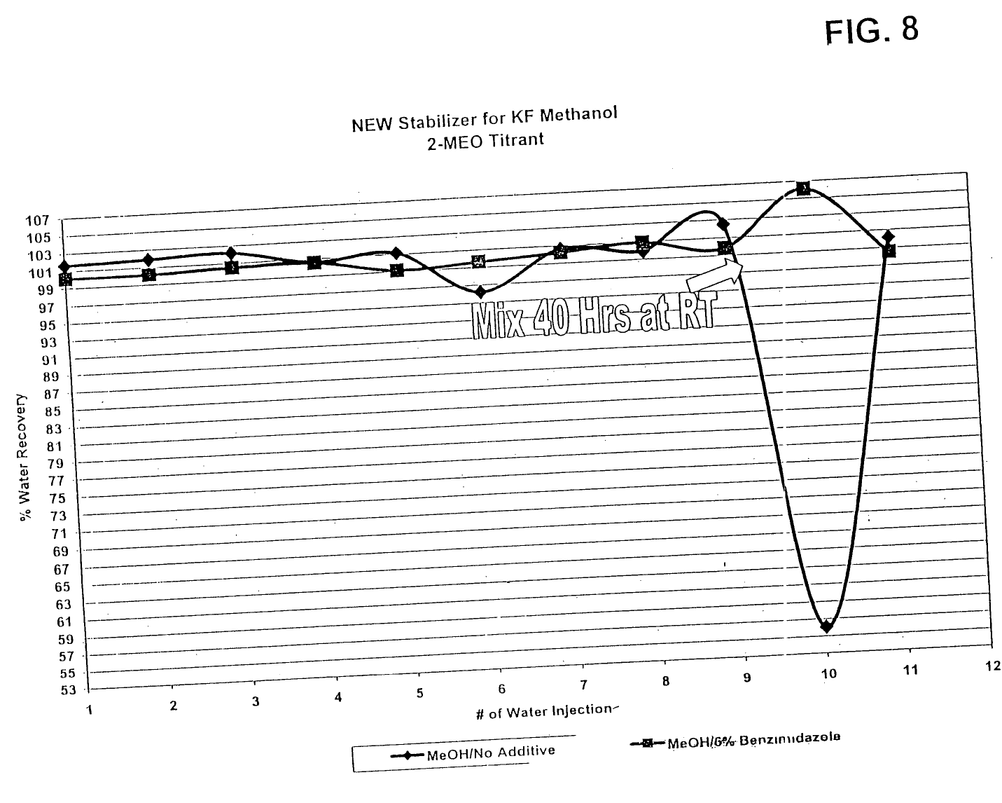

The same procedure of Examples 1-3 is repeated except that the methanol solvent charged to the reaction vessel contains 6% by weight of benzimidazole as the stabilizer. Also, the time period between the 9^{th} and 10^{th} injections is extended to 40 hours. The water recovery values are shown in FIG. 8 by the lines with the square-shaped points and the data from the previous run using no stabilizer in the methanol solvent is included for comparison purposes. It is seen that the stabilizer eliminates the false low water recovery values for titrations conducted after both a 1 hour and 40 hour period of inactivity.

### Example 10

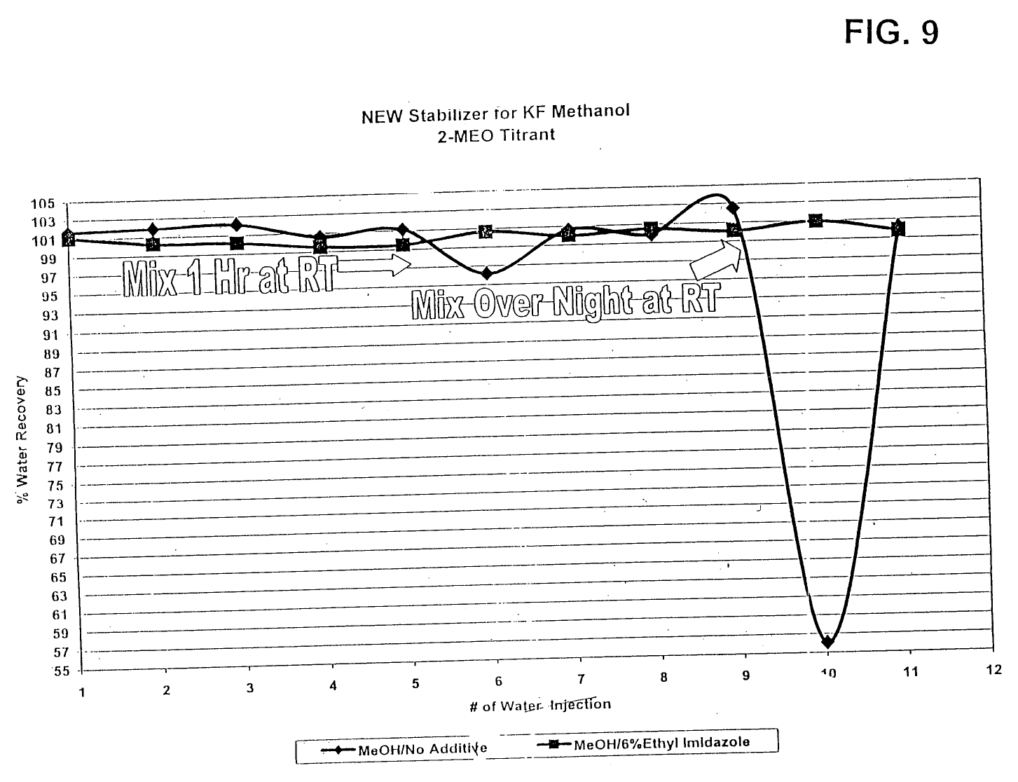

The same procedure of Examples 1-3 is repeated except that the methanol solvent charged to the reaction vessel contains 6% by weight of ethylimidazole as the stabilizer. The water recovery values are shown in FIG. 9 by the lines with the square-shaped points and the data from the previous run using no stabilizer in the methanol solvent is included for comparison purposes. It is seen that the stabilizer eliminates the false low water recovery values for titrations conducted after both a 1 hour and 16 hour period of inactivity.

### Example 11

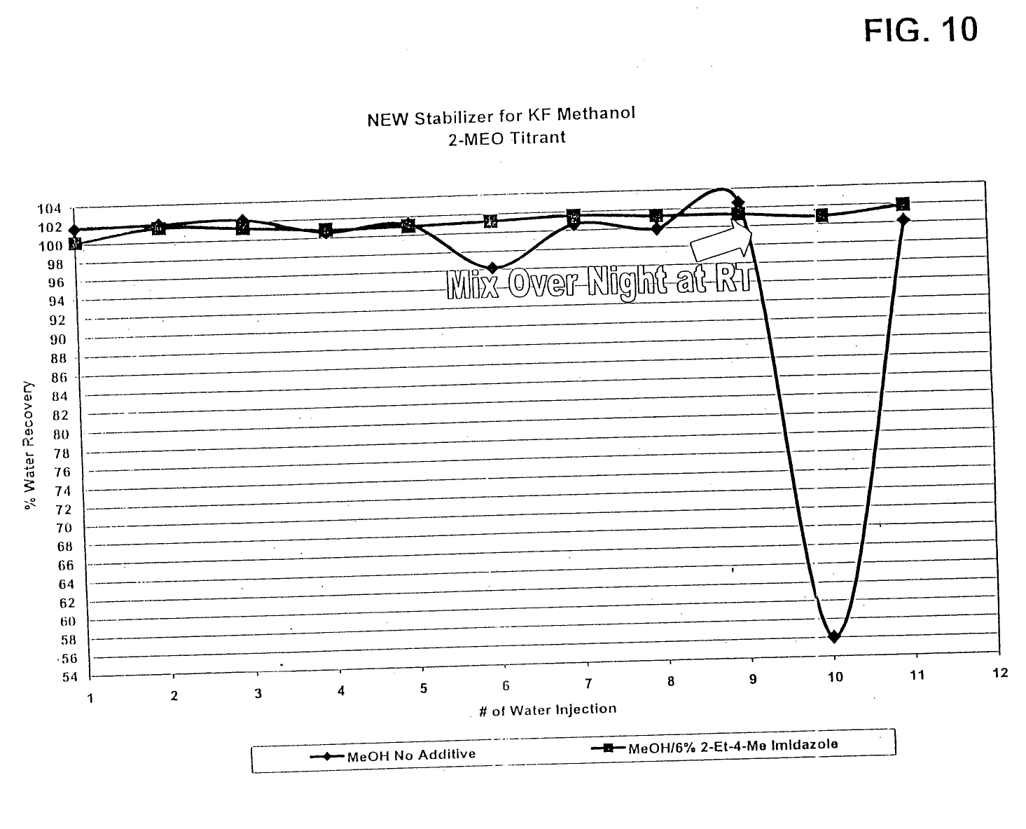

The same procedure of Examples 1-3 is repeated except that the methanol solvent charged to the reaction vessel contains 6% by weight of 2-ethyl-4-methylimidazole as the stabilizer. The water recovery values are shown in FIG. 10 by the lines with the square-shaped points and the data from the previous run using no stabilizer in the methanol solvent is included for comparison purposes. It is seen that the stabilizer eliminates the false low water recovery values for titrations conducted after both a 1 hour and 16 hour period of inactivity.

### Example 12

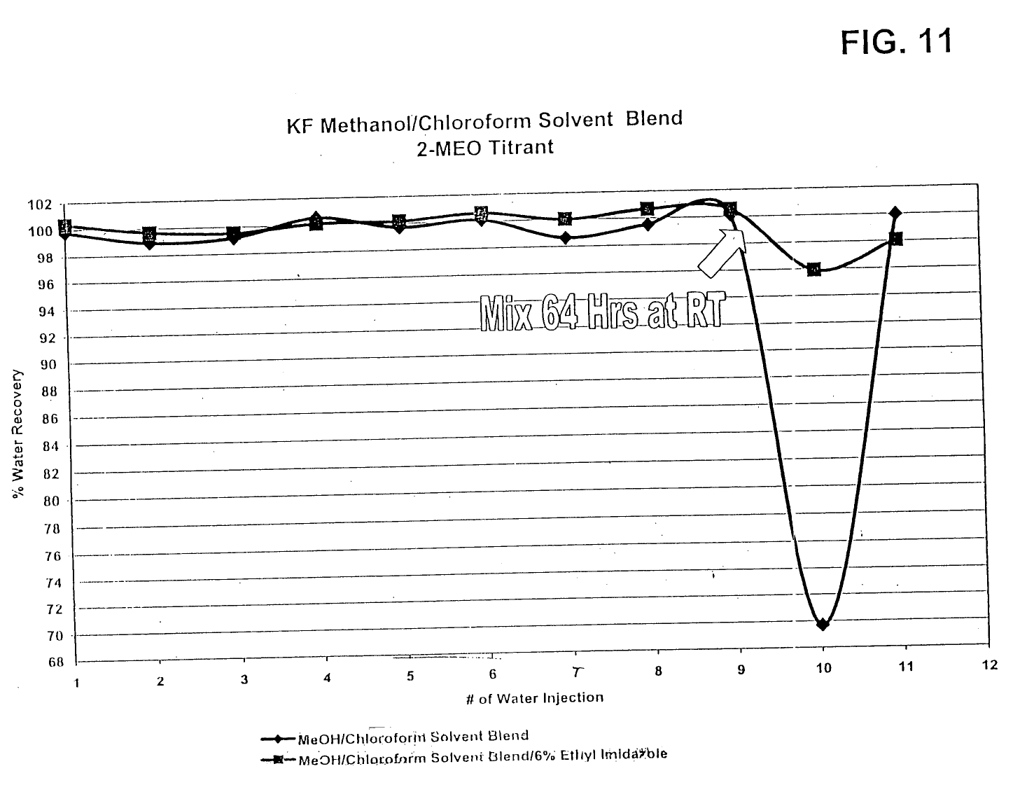

Using the same known volumetric Karl-Fischer titration instrument discussed above, Karl-Fischer titrations are performed as follows. A 60 wt % methanol: 40 wt % chloroform solvent, for example of 50 to 100 ml total volume, is charged to the reaction vessel. The solvent is zeroed-out by titration so that it has a zero water value. Nine pure water samples, 0.0200 grams each, are successively injected into the reaction vessel and titrated with titrant as identified in Example 1. The instrument is then left inactive for 64 hours. A further pure water sample of 0.0200 grams is injected and titrated in the same manner and then a subsequent identical sample is injected and titrated. Thus, 11 samples total are injected and titrated. The water recovery % by titration for each sample is determined and recorded in FIG. 11 (shown by the line with diamond-shaped points). The water recovery values determined are at or near the expected 100% water recovery value for the first nine samples the 10^{th} sample, after the 64 hours inactivity, has a false low value of about 70%. The subsequent sample shows the true 100% value.

### Example 13

The same procedure of Example 12 is repeated except that the methanol/chloroform solvent charged to the reaction vessel additionally contains 6% by weight of ethylimidazole as a stabilizer. The water recovery values are shown also in FIG. 11 by the line with the square-shaped points. It is seen that the stabilizer lessens the false low water recovery value of the 10^{th} injection, after 64 hours inactivity.

### Example 14

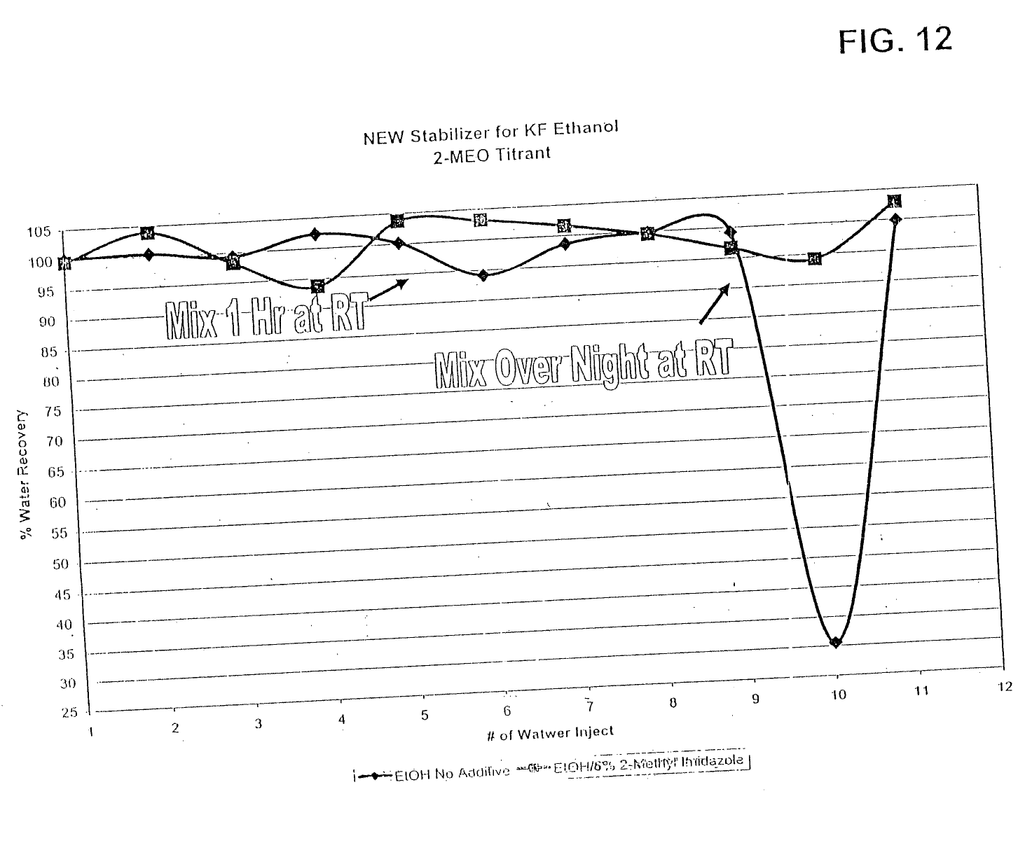

Using the same known volumetric Karl-Fischer titration instrument discussed above, Karl-Fischer titrations are performed as follows. An ethanol solvent, for example of 50 to 100 ml total volume, is charged to the reaction vessel. The solvent is zeroed-out by titration so that it has a zero water value. Five pure water samples, 0.0200 grams each, are successively injected into the reaction vessel and titrated with the same titrant as in Example 1. The instrument is then left inactive for one hour. A further pure water sample of 0.0200 grams is injected and titrated in the same manner and then three further identical samples are injected and titrated. The instrument is left inactive overnight (16 hours) and the next morning another identical water sample is titrated in the same manner and then a further sample immediately following. Thus, 11 samples total are injected and titrated. The water recovery % by titration for each sample is determined and recorded in FIG. 12 (shown by the line with diamond-shaped points). The water recovery values determined are at or near the expected 100% water recovery value except the 6^{th} sample injected immediately after the 1 hour inactivity has a false low value about 94% and the 10^{th} sample injected immediately after the overnight activity has a false very low value about 30%.

### Example 15

The same procedure of Example 14 is repeated except that the ethanol solvent charged to the reaction vessel additionally contains 6% by weight of 2-methylimidazole as a stabilizer. The water recovery values are shown also in FIG. 12 by the line with the square-shaped points. It is seen that the stabilizer eliminates the false low water recovery value of the 6^{th} injection, after one hour inactivity, and significantly lessens the extent of the false low water recovery value of the 10^{th} injection, after overnight (about 16 hours) inactivity.

The entire disclosures of all applications, patents and publications, cited herein and of corresponding U.S. Provisional Application Ser. No. 60/548,875, filed Mar. 2, 2004 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method for avoiding false low water determinations in a volumetric Karl-Fischer water determination which comprises:
in a method wherein an alcohol-containing solvent is charged to a reaction vessel and a titrant comprising iodine, sulfur dioxide, a titrant solvent and, a titrant base compound, is subsequently titrated into the reaction vessel to:
zero-out the water contained in the alcohol-containing solvent charged to the reaction vessel;
determine the water content of a standard of known water concentration charged into the reaction vessel containing alcohol-containing solvent; and/or
determine the water content of a sample charged into the reaction vessel containing alcohol-containing solvent;
including an amine group-containing compound, separate from the titration, as a stabilizer in the alcohol-containing solvent charged to the reaction vessel.

2. The method of claim 1, wherein the stabilizer is provided in the solvent charged to the reaction vessel in an amount of 2 to 15% by weight based on the total resulting composition of solvent and stabilizer.

3. The method of claim 1, wherein the stabilizer is provided in the solvent before the solvent is charged into the reaction vessel.

4. The method of claim 1, wherein the stabilizer is provided in the solvent after the solvent is charged into the reaction vessel.

5. The method of claim 1, wherein, after the stabilizer is provided, and, after at least one titration step to:
zero-out the water contained in the alcohol-containing solvent charged to the reaction vessel;
determine the water content of a standard of known water concentration charged into the reaction vessel containing alcohol-containing solvent; and/or
determine the water content of a sample charged into the reaction vessel containing alcohol-containing solvent;
the reaction vessel is maintained inactive for at least one hour and then a further titration step to:
zero-out the water contained in the alcohol-containing solvent charged to the reaction vessel;
determine the water content of a standard of known water concentration charged into the reaction vessel containing alcohol-containing solvent; and/or
determine the water content of a sample charged into the reaction vessel containing alcohol-containing solvent;
is conducted.

6. The method of claim 1, wherein the stabilizer is an alkanolamine, a substituted or unsubstituted pyridine or derivative thereof, a substituted or unsubstituted imidazole or derivative thereof or a mixture of any of the above.

7. The method of claim 2, wherein the stabilizer is an alkanolamine, a substituted or unsubstituted pyridine or derivative thereof, a substituted or unsubstituted imidazole or derivative thereof or a mixture of any of the above.

8. The method of claim 1, wherein the stabilizer is: mono-, di- or tri-ethanolamine; mono-, di- or tri-isopropanolamine; imidazole; 2-methylimidazole; 2-ethyl-4-methylimidazole; 2-ethylimidazole; phenylimidazole; benzimidazole; pyridine; or a mixture thereof.

9. The method of claim 1, wherein the alcohol-containing solvent charged to the reaction vessel is methanol, ethanol, a glycol ether, 2-methoxyethanol, a mixture of a primary amount of one of these with another solvent or a mixture of any of the above.

10. The method of claim 1, wherein the alcohol-containing solvent charged to the reaction vessel is methanol or a mixture of a primary amount of methanol with another solvent.

11. The method of claim 1, wherein the alcohol-containing solvent charged to the reaction vessel is a mixture of a primary amount of methanol mixed with a chlorinated solvent.

12. The method of claim 1, wherein the alcohol-containing solvent charged to the reaction vessel is a mixture of a primary amount of methanol mixed with chloroform.

13. The method of claim 1, wherein the titrant comprises iodine, sulfur dioxide, an alcohol solvent and an amine-containing base compound.

14. The method of claim 1, wherein the titrant comprises iodine, sulfur dioxide, a glycol ether solvent and an imidazole base compound.

15. The method of claim 1, wherein the titrant contains 1 to 10 mg/ml of iodine.

16. The method of claim 1, wherein the titrant contains 1 to 5 mg/ml of iodine.

17. The method of claim 1, wherein the endpoint of titrations is determined potentiometrically.

18. A solvent composition for use in a volumetric Karl-Fischer titration, which comprises an alcohol-containing solvent and 2 to 15% by weight, based on the total composition, of an amine-containing compound as stabilizer.

19. The solvent composition of claim 18, which comprises 3 to 7% by weight, based on the total composition, of the stabilizer.

20. The composition of claim 18, wherein the stabilizer is an alkanolamine, a substituted or unsubstituted pyridine or derivative thereof, a substituted or unsubstituted imidazole or derivative thereof or a mixture of any of the above.

21. The composition of claim 18, wherein the stabilizer is: mono-, di- or tri-ethanolamine; mono-, di- or tri-isopropanolamine; imidazole; 2-methylimidazole; 2-ethyl-4-methylimidazole; 2-ethylimidazole; phenylimidazole; benzimidazole; pyridine; or a mixture thereof.

22. The composition of claim 18, wherein the alcohol-containing solvent charged to the reaction vessel is methanol, ethanol, a glycol ether, 2-methoxyethanol, a mixture of a primary amount of one of these with another solvent or a mixture of any of the above.

23. The composition of claim 21, wherein the alcohol-containing solvent charged to the reaction vessel is methanol, ethanol, a glycol ether, 2-methoxyethanol, a mixture of a primary amount of one of these with another solvent or a mixture of any of the above.

24. The composition of claim 18, wherein the alcohol-containing solvent charged to the reaction vessel is methanol or a mixture of a primary amount of methanol with another solvent.

25. The composition of claim 21, wherein the alcohol-containing solvent charged to the reaction vessel is methanol or a mixture of a primary amount of methanol with another solvent.

26. The composition of claim 18, wherein the alcohol-containing solvent charged to the reaction vessel is a mixture of a primary amount of methanol mixed with a chlorinated solvent.

27. The composition of claim 18, wherein the alcohol-containing solvent charged to the reaction vessel is a mixture of a primary amount of methanol mixed with chloroform.
